(19) United States
(12) Patent Application Publication
(10) Pub. No.: US 2024/0376075 A1
(43) Pub. Date: Nov. 14, 2024

(54) POLYMORPHIC FORMS OF AURORA A SELECTIVE INHIBITORS AND USES THEREOF

(71) Applicant: Jacobio Pharmaceuticals Co., Ltd., Beijing (CN)

(72) Inventors: Haijun LI, Beijing (CN); Guiqun YANG, Beijing (CN); Wei LONG, Beijing (CN); Yanping WANG, Beijing (CN)

(21) Appl. No.: 18/292,360

(22) PCT Filed: Jul. 27, 2022
(86) PCT No.: PCT/CN2022/108128

§ 371 Date: Jan. 26, 2024

(30) Foreign Application Priority Data
Jul. 28, 2021 (WO) PCT/CN2021/108776

(51) Int. Cl. C07D 401/14; A61K 31/4545

(52) U.S. Cl. CPC C07D 401/14; A61K 31/4545

(57) ABSTRACT

Provided herein are salts of the compound 1 and new poly morph forms thereof, pharmaceutical composition including the compound 1 and new polymorph forms thereof, and methods of preparing them and using them.

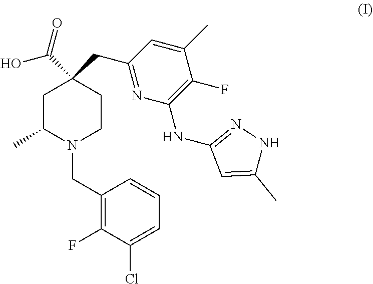

### CROSS REFERENCE TO RELATED APPLICATIONS

The application claims the benefit of PCT application Ser. No. PCT/CN2021/108776 filed on Jul. 28, 2021; the contents of which are hereby incorporated herein by reference in its entirety.

### FIELD OF THE INVENTION

The present invention relates to salts the Aurora A inhibitor and new polymorphic forms thereof, processes for preparing these salts and new polymorphic forms, pharmaceutical compositions thereof, and use of them in the treatment of an Aurora A mediated disease.

### BACKGROUND OF THE INVENTION

Aurora kinases are a family of serine/threonine kinases and are key regulators of mitosis. There are three human homologs of Aurora kinases, A, B, and C, of which Aurora A has been implicated in cancers of diverse histological origin and may possess oncogenic properties when overexpressed.

Aurora-A localizes to centrosomes/spindle poles and is required for spindle assembly, whereas Aurora-B is a chromosome passenger protein required for phosphorylation of histone H3, chromosome segregation and cytokinesis. Aurora-A and -B are both overexpressed in a wide range of different human tumours. Additionally, certain Aurora B inhibitors and Aurora A/B dual inhibitors in clinical development have been reported as presenting neutropenia and bone marrow cytotoxicity in patients while certain relatively selective Aurora A inhibitors in clinical development did not show these disorders. Therefore, it is desirable to selectively inhibit Aurora A and reduce or avoid Aurora B or Aurora A/B dual inhibition. As such, selective Aurora A inhibition may be useful for cancer therapy.

Therefore, there remains a need to provide alternative Aurora A inhibitors for treatment of cancer. Also, there remains a need to provide selective Aurora A inhibitors that reduce or avoid Aurora B or Aurora A/B dual inhibition. Accordingly, the present invention provides certain inhibitors of Aurora A which may be useful for treating cancer. The compounds of the present invention fulfill the need of small molecules in order to inhibit the activity of Aurora A.

Many pharmaceutically active organic compounds can crystallize in more than one type of three-dimensional crystal structure. That is, the compounds may crystallize in different crystalline forms. This phenomenon (identical chemical structure but different crystalline structure) is referred to as polymorphism, and the species having different molecular structures are referred to as polymorphs.

Polymorphs of a particular organic pharmaceutical compound may have different physical properties, such as solubility, stability, and hygroscopicity due to their distinct three-dimensional crystal structures. However, it is generally not possible to predict whether a particular organic compound will form different crystalline forms, let alone predict the structure and properties of the crystalline forms themselves. The discovery of a new crystalline or polymorph form of a pharmaceutically useful compound may provide a new opportunity for improving the overall characteristics of a pharmaceutical product. It enlarges the repertoire of materials that a formulation scientist has available for designing. It may be advantageous when this repertoire is enlarged by the discovery of new polymorphs of a useful compound.

### DESCRIPTIONS OF THE INVENTION

One of the objects of the present invention is to provide a salt form of a Compound 1, preferably its potassium salt, sodium salt, p-toluenesulfonate, tert-butylamine salt, hydrochloride, or mesylate, and polymorph forms thereof.

The Compound 1 described herein refers to a compound with the following structure:

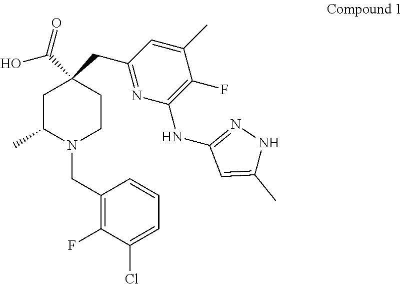

The “salts” described herein include pharmaceutically acceptable salts as well as pharmaceutically unacceptable salts. It is not preferable to apply the pharmaceutically unacceptable salts to patients, but these salts can be used to provide pharmaceutical intermediates and bulk pharmaceutical forms. For example the salt maybe selected from the group consisting of hydrochloride, sulfate, bisulfate, nitrate, hydrobromide, hydriodate, carbonate, bicarbonate, sulfite, bisulfite, pyrosulfate, monohydrogen phosphate, dihydrogen phosphate, perchlorate, persulfate, semi-sulfate, bisulfate, thiocyanate, phosphate, pyrophosphate, metaphosphate, formate, acetate, propionate, butyrate, benzoate, malonate, succinate, pyruvate, mesylate, ethanesulfonate, propanesulfonate, citrate, 4-nitrobenzoate, benzenesulfonate, p-toluenesulfonate, 1,2-ethanedisulfonate, β-naphthalenesulfonate, malate, propiolate, 2-butynoate, 2-hydroxy-ethanesulfonate, vinyl acetate, tartaric acid salt, fumarate, isethionate, maleate, lactate, lactobionate, pamoate, salicylate, galactarate, glucoheptonate, Mandelate, 1,2-ethanedisulfonate, oxalate, trifluoroacetate, triflate, adipate, suberate, sebacate, butyne-1,4-Diacid, Hexyne-1,6-Diacid, Glycolate, Alginate, Ascorbate, Aspartate, Glutamate, 2-Phenoxybenzyl acid salt, 2-(4-hydroxybenzoyl)benzoate, acetoacetate, 2-hydroxyethanesulfonate, borate, chlorobenzoate, camphorate, itaconate, camphorsulfonate, methylbenzoate, dinitrobenzoate, sulfamate, galacturonate, cyclopentylpropionate, lauryl sulfate, acrylate, cyclopentane Pentane Propionate, Glycerophosphate, Methoxybenzoate, Digluconate, Gluconate, Heptanoate, Caproate, Trimethyl Acetate, Glucuronate, Lauric Acid salt, phthalate, phenylacetate, lauryl sulfate, 2-acetoxybenzoate, nicotinate, cinnamate, oleate, palmitate, pectinate, Phthalate, Glutarate, Hydroxymaleate, Hydroxybenzoate, Phenylacetate, 3-Hydroxy-2-naphthoate, 3-Phenylpropionate, Isobutyric Acid Salt, pivalate, picrate, stearate, 2,2-dichloroacetate, acylated amino acid salt, alginate, 4-acetamidobenzenesulfonate, caprate, cholic acid salt, octanoate, pelargonate, cyclamate, phthalate, cysteine hydrochloride, sorbate, glycinate hydrochloride, 1,5-naphthalene disulfonate, xylene sulfonate, Cystine dihydrochloride, undecanoate, polyvinylsulfonate, sulfosalicylate, phenylbutyrate, 4-hydroxybutyrate, polyvinyl sulfate, naphthalene-1-sulfonate, and pentamate.

Compound 1 can form a salt with one or two equivalents of acid (abbreviated as mono-salt or di-salt), for example, its hydrobromide can be monohydrobromide or dihydrobromide. Generally, when preparing a salt form of Compound 1, the corresponding mono- or di-salt can be generated by controlling the molar ratio of the compound to the corresponding acid. However, it is difficult to completely control the equivalent of 1:1 or 1:2 during actual operation, and in large-scale preparations, due to the locally excessive presence of acid or Compound 1, a mixture of a mono-salt and a di-salt may be formed. Because the physical and chemical properties of a mono-salt are different from those of a di-salt, the formation of this mixture will result in non-uniform properties of the final product. Therefore, it will bring great convenience to the preparation and production if the formation of a certain salt type is relatively easily controlled, and final products with uniform qualities can be obtained more easily. The inventor has discovered by accident that for potassium salt, sodium salt, p-toluenesulfonate, tert-butylamine salt, hydrochloride, and mesylate of Compound 1, a mono-salt can be formed in high yields at a molar ratio of the compound to the corresponding acid of slightly less than 1:1, such as 1:1.1 (acid excess), so the scale-up process is simplified and the efficiency is improved.

As described herein, compared with Compound 1, some salt forms of Compound 1, such as potassium salt, sodium salt, p-toluenesulfonate, tert-butylamine salt, hydrochloride or mesylate, have more or less improved water solubility, and some polymorphs of these salt forms (especially sodium salt crystalline form, potassium salt crystalline form, hydrochloride crystalline form, etc.) have properties such as high stability, low moisture absorption, which is beneficial to the production and preparation of Compound 1, and is of great significance to its final marketization.

The preparation of compound 1 refers to the preparation method of Example 78 in the application with the application No. PCT/CN2021/073169. The entire content of this application is incorporated herein.

The present invention provides a method for preparing a salt of a compound 1, comprising the step of forming a salt of the compound 1 with a counter ion in a solvent. The present invention further provides a method for preparing the polymorph form of the salt of the compound 1, comprising: taking a certain amount of the compound 1, adding an appropriate amount of corresponding solvent, adding counter ions, crystallization, centrifugation and drying to obtain the corresponding polymorph form of salt of the compound 1. In some embodiments, the above-mentioned counter ion may be sodium ion, potassium ion, p-toluenesulfonic acid, tert-butylamine acid, hydrochloric acid, methanesulfonic acid, and the like. In some embodiments, the crystalline form of the salt is the crystalline form of sodium salt, the crystalline form of potassium salt, the crystalline form of p-toluenesulfonate salt, the crystalline form of tert-butylamine salt, the crystalline form of hydrochloride salt, the crystalline form of methanesulfonic acid Salt crystal forms, etc. In some embodiments, the solvent may be selected from hydrocarbon solvents, ether solvents, alcohol solvents, ester solvents, ketone solvents, nitrile solvents, halogenated hydrocarbon solvents, nitrogen-containing solvents, water, dimethyl one or more of the sulfoxides. The hydrocarbon solvents include but are not limited to cyclohexane, n-heptane, p-xylene; the ether solvents include but are not limited to tetrahydrofuran, diethyl ether, propylene glycol methyl ether, methyl tert-butyl ether, isopropyl ether or 1,4-dioxane; the alcohol solvents include but are not limited to methanol, ethanol, isopropanol, n-propanol, isoamyl alcohol or trifluoroethanol; the ester solvents include but are not limited to ethyl acetate, isopropyl acetate or butyl acetate; the ketone solvents include but are not limited to acetone, acetophenone, 4-methyl-2-pentanone; the nitrile solvents include but are not limited to acetonitrile and propionitrile; the Halogenated hydrocarbon solvents include but are not limited to methyl chloride, dichloromethane, 1,2-dichloroethane, chloroform or carbon tetrachloride; the nitrogen-containing solvents include but are not limited to nitromethane, N,N-dichloromethane Methylformamide, N,N-dimethylacetamide.

The presently disclosed invention is especially directed to a compound of Formula I, which is the potassium salt of compound 1, namely potassium (2R,4R)-1-(3-chloro-2-fluorobenzyl)-4-((5-fluoro-4-methyl-6-((5-methyl-1H-pyrazol-3-yl)amino)pyridin-2-yl)methyl)-2-methylpiperidine-4-carboxylate, approximately pure polymorph forms thereof, or pharmaceutical acceptable salts thereof. The polymorph forms of the compound of Formula I have great solubility, chemical stability, and making them preferable for application.

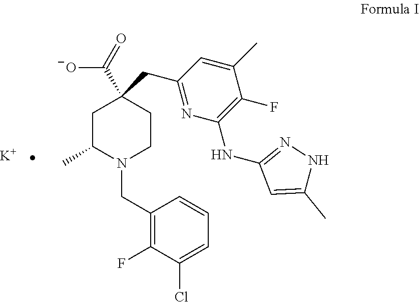

The compound of Formula I of the present invention exists in one or more polymorph forms. In one embodiment, the polymorph forms is selected from the group consisting of polymorph form I, polymorph form II, and polymorph form III. In another embodiment, the compound of Formula I may be anhydrous, or may contain varying amounts of water or one or more solvents.

Each polymorph form may be characterized by analytical method well known in the field of the pharmaceutical industry for characterizing solids. Such methods comprise but are not limited to X-ray powder diffraction (XRPD), differential scanning calorimetry analysis (DSC), thermogravimetric analysis (TGA), dynamic vapor sorption (DVS), fourier transform infrared spectrometer (FT-IR), and high performance liquid chromatography (HPLC). Each polymorph form may be characterized by one of the aforementioned analytical methods or by combining two or more them.

In one aspect, the present invention provides polymorph form I of the compound of Formula I.

In some embodiments, polymorph form I is a monohydrate.

In some embodiments, polymorph form I, when characterized by X-ray powder diffraction, has an X-Ray diffraction pattern with peaks at diffraction angles 2θ of at least one of 4.7°±0.2°, 9.3°±0.2°, 14.1°±0.2°.

In some embodiments, polymorph form I, when characterized by X-ray powder diffraction, has an X-Ray diffraction pattern with peaks at diffraction angles 2θ of 4.7°±0.2°, 9.3°±0.2°, 14.1°±0.2°, 22.1°±0.2° and 26.4°±0.2°.

In some embodiments, polymorph form I, when characterized by X-ray powder diffraction, has an X-Ray diffraction pattern with peaks at diffraction angles 2θ of 4.7°±0.2°, 9.3°±0.2°, 14.1°±0.2°, 15.9°±0.2°, 17.9°±0.2°, 22.1°±0.2°, 26.4°±0.2°, 32.2°±0.2° and 38.0°±0.2°.

In some embodiments, polymorph form I, when characterized by X-ray powder diffraction, has an X-Ray diffraction pattern with peaks at diffraction angles 2θ of 4.7°±0.2°, 9.3°±0.2°, 14.1°±0.2°, 15.9°±0.2°, 17.9°±0.2°, 19.5°±0.2°, 22.1°±0.2°, 23.5°±0.2°, 24.6°±0.2°, 25.0°±0.2°, 26.4°±0.2°, 32.2°±0.2°, 35.6°±0.2° and 38.0°±0.2°.

In some other embodiments, the X-ray powder diffraction pattern of polymorph form I is substantially shown as in FIG. 1.

In some other embodiments, the differential scanning calorimetry analysis spectrum of the polymorph form I is substantially characterized as in FIG. 2, when measured at a temperature in the range of from 0 to 300° C., and a heating rate of about 10° C./min.

In some embodiments, the hygroscopicity of polymorph form I is ≤1%, more preferably ≤0.5%, most preferably ≤0.2%. In some embodiments, the polymorph form I is no hygroscopicity.

In one aspect, the present invention provides polymorph form II of the compound of Formula I.

In some embodiments, polymorph form II is a solvate.

In some embodiments, polymorph form II is a hydrate.

In some embodiments, polymorph form II, when characterized by X-ray powder diffraction, has an X-Ray diffraction pattern with peaks at diffraction angles 2θ of 8.7°±0.2°, 11.1°±0.2° and 15.8°±0.2°.

In some embodiments, polymorph form II, when characterized by X-ray powder diffraction, has an X-Ray diffraction pattern with peaks at diffraction angles 2θ of 8.7°±0.2°, 11.1°±0.2°, 15.8°±0.2°, 16.2°±0.2° and 21.1°±0.2°.

In some embodiments, polymorph form II, when characterized by X-ray powder diffraction, has an X-Ray diffraction pattern with peaks at diffraction angles 2θ of 8.7°±0.2°, 11.1°±0.2°, 14.1°±0.2°, 15.8°±0.2°, 16.2°±0.2°, 18.8°±0.2°, 21.1°±0.2°, 25.0°±0.2° and 30.1°±0.2°.

In some embodiments, polymorph form II, when characterized by X-ray powder diffraction, has an X-Ray diffraction pattern with peaks at diffraction angles 2θ of 4.7°±0.2°, 8.7°±0.2°, 9.4°±0.2°, 11.1°±0.2°, 14.1°±0.2°, 15.8°±0.2°, 16.2°±0.2°, 18.8°±0.2°, 21.1°±0.2°, 22.1°±0.2°, 25.0°±0.2°, 30.1°±0.2° and 32.5°±0.2°.

In some other embodiments, the X-ray powder diffraction pattern of polymorph form II is substantially shown as in FIG. 3.

In some other embodiments, the differential scanning calorimetry analysis spectrum of the polymorph form II is substantially characterized as in FIG. 4.

In one aspect, the present invention provides polymorph form III of the compound of Formula I.

In some embodiments, polymorph form III, when characterized by X-ray powder diffraction, has an X-Ray diffraction pattern with peaks at diffraction angles 2θ of 5.1°±0.2°, 10.1°±0.2° and 12.7°±0.2°.

In some embodiments, polymorph form III, when characterized by X-ray powder diffraction, has an X-Ray diffraction pattern with peaks at diffraction angles 2θ of 5.1°±0.2°, 10.1°±0.2°, 12.7°±0.2°, 21.0°±0.2° and 24.9°±0.2°.

In some embodiments, polymorph form III, when characterized by X-ray powder diffraction, has an X-Ray diffraction pattern with peaks at diffraction angles 2θ of 5.1°±0.2°, 10.1°±0.2°, 12.7°±0.2°, 14.8°±0.2°, 20.0°±0.2°, 21.0°±0.2°, 23.0°±0.2°, 24.9°±0.2° and 25.5°±0.2°.

In some embodiments, polymorph form III, when characterized by X-ray powder diffraction, has an X-Ray diffraction pattern with peaks at diffraction angles 2θ of 5.1°±0.2°, 10.1°±0.2°, 12.7°±0.2°, 14.8°±0.2°, 15.2°±0.2°, 15.5°±0.2°, 17.7°±0.2°, 19.7°±0.2°, 20.0°±0.2°, 21.0°±0.2°, 24.9°±0.2° and 25.5°±0.2°.

In some other embodiments, the X-ray powder diffraction pattern of polymorph form III is substantially shown as in FIG. 5.

Polymorph Form I, II or III disclosed herein can have a purity of ≥85%, ≥95%, ≥99%, or even ≥99.5%.

The amorphous form of Compound of Formula I provide a further embodiment of the invention.

The polymorph Form I described herein exhibits surprisingly advantageous thermodynamic stability when compared to the polymorphs Form II and Form III at room temperature. The polymorph Form I is therefore advantageous during the process of preparation, transportation, storage and preservation.

In still another aspect, the present invention provides processes for preparing a polymorph form of the compound of Formula I according to the above aspect of the invention is provided.

In one embodiment, the invention provides a process for the preparation of the polymorph form I, comprising:

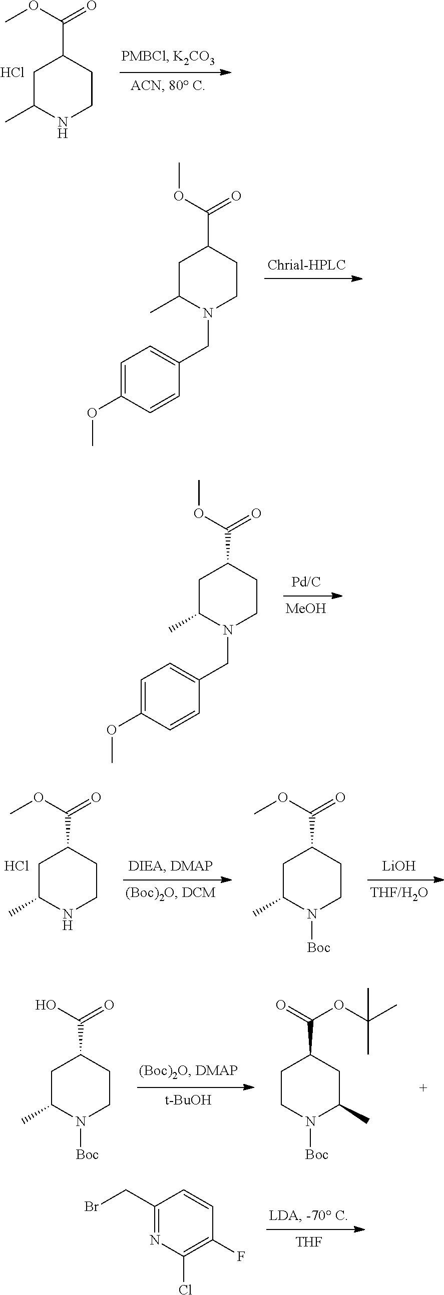

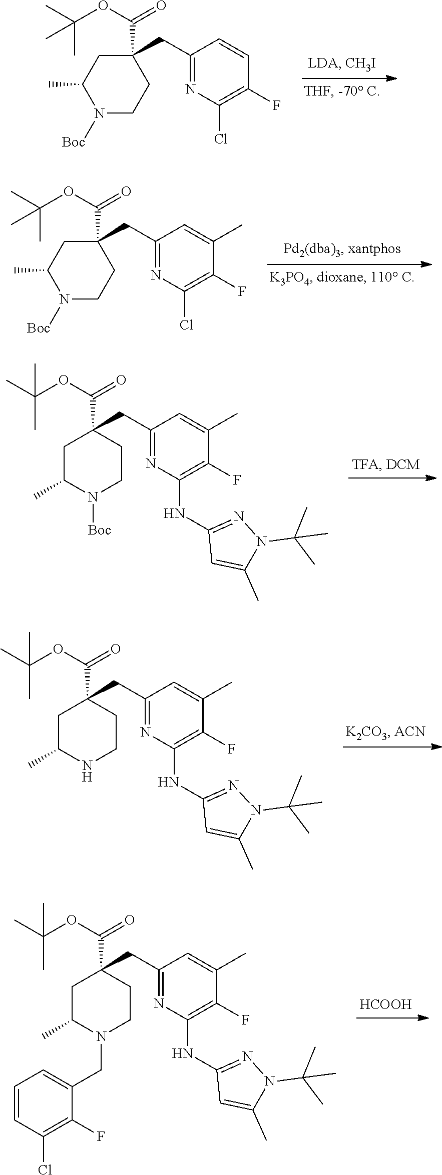

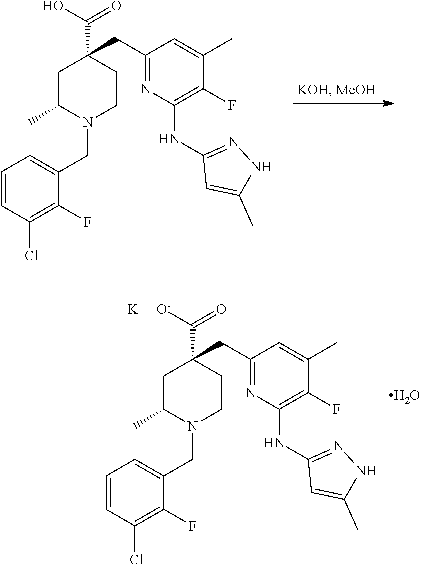

In one embodiment, the polymorph form I of potassium (2R,4R)-1-(3-chloro-2-fluorobenzyl)-4-((5-fluoro-4-methyl-6-((5-methyl-1H-pyrazol-3-yl)amino)pyridin-2-yl)methyl)-2-methylpiperidine-4-carboxylate was obtained from via vapor-solid diffusion crystallization comprising placing an amorphous of the compound of Formula I in ethanol or in dichloromethane atmosphere for a period time. In one embodiment, the time is 4 days.

In one embodiment, the polymorph form I of potassium (2R,4R)-1-(3-chloro-2-fluorobenzyl)-4-((5-fluoro-4-methyl-6-((5-methyl-1H-pyrazol-3-yl)amino) pyridin-2-yl)methyl)-2-methylpiperidine-4-carboxylate was obtained from polymorph form II dried under 40° C. for overnight.

In one embodiment, the invention provides a process for the preparation of the polymorph form I which comprises suspending an amorphous of the compound of Formula I in a suitable solvent, and then isolating the resulting product. In one embodiment, the solvent is selected from isopropanol, acetone, ethyl acetate, or acetonitrile.

In a further embodiment the invention provides a process for the preparation of the polymorph form II choose from evaporation crystallization which comprises dissolving the polymorph form I of the compound of Formula I in a suitable solvent, evaporating at RT, and then isolating the resulting product. In one embodiment, the solvent is selected from acetone/water or tetrahydrofuran (THF)/water. In one embodiment, the volume radio between acetone and water is 5:2. In one embodiment, the volume radio between THF and water is 5:2 or 10:3.

In one embodiment, the polymorph form II was obtained from via vapor-solid diffusion crystallization comprising placing an amorphous of the compound of Formula I in water atmosphere for a period time. In one embodiment, the time is 4 days.

The amorphous form of potassium (2R,4R)-1-(3-chloro-2-fluorobenzyl)-4-((5-fluoro-4-methyl-6-((5-methyl-1H-pyrazol-3-yl)amino) pyridin-2-yl)methyl)-2-methylpiperidine-4-carboxylate can be prepared using the techniques exemplified herein.

In one embodiment, amorphous form of potassium (2R,4R)-1-(3-chloro-2-fluorobenzyl)-4-((5-fluoro-4-methyl-6-((5-methyl-1H-pyrazol-3-yl)amino) pyridin-2-yl)methyl)-2-methylpiperidine-4-carboxylate was obtained from via vacuum concentration in methanol/isopropyl ether/acetonitrile system; or obtained via evaporation in trifluoroethanol.

Crystallization used herein to isolate a polymorph form of the compound of formula I as set forth above can be carried out in a single solvent, or a mixture of solvents.

Suitable solvents for the crystallization to achieve isolation of a polymorph can be chosen from, but are not limited to, low carbon alcohols, ketones, ethers, esters, halogenated hydrocarbons, alkanes, halogenated benzene, aliphatic nitrile, and other aromatic solvents. As non-limiting example, the solvent for the crystallization of the compound of formula I can be chosen from isopropanol, ethyl acetate, 50% ethanol, water, N,N-dimethylformamide, methanol, ethanol, acetone, and propanol.

The crystallization of the polymorph forms of the present invention can be conducted by any conventional techniques well-known in the art. Such crystallization techniques may include, without limitation, one or more of the following: precipitation, evaporation, slurrying, cooling, diffusion, milling, addition of anti-solvents and polymer template, or any combination thereof.

As disclosed herein, crystallization may be done with or without seed crystals.

The individual crystalline forms disclosed herein can develop under specific conditions dependent on the particular thermodynamic and equilibrium properties of the crystallization process. Therefore, any persons of ordinary skill in the art of polymorphism in this area know that the formed crystals are a consequence of the kinetic and thermodynamic properties of the crystallization process. Under certain conditions (e.g., solvent, temperature, pressure, and concentration of the compound of this invention), a particular crystalline form may be more stable than another crystalline form (or in fact more stable than any other crystalline forms). However, the relatively low thermodynamic stability of particular crystals may have advantageous kinetics. Additional factors other than kinetics, such as time, impurity distribution, stirring, and the presence or absence of seed crystals, etc., may also affect the crystalline form.

In another aspect, the present invention provides a pharmaceutical composition comprising any one or more of the salt forms or crystalline forms described herein and a pharmaceutically acceptable carrier or diluent. In some embodiments, the present invention provides a pharmaceutical composition comprising a therapeutically effective amount of at least one polymorph form of the compound of Formula I disclosed herein and at least one pharmaceutically acceptable excipient, adjuvant, or carrier.

The term “therapeutically effective amount” refers to the amount of a compound that, when administered to a subject for treating a disease, or at least one of the clinical symptoms of a disease or disorder, is sufficient to affect such treatment for the disease, disorder, or symptom. The “therapeutically effective amount” can vary depending on the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combined active ingredient for the effective treatment of a disease, a disorder or a condition.

In some embodiments, the pharmaceutical composition comprises 0.01 wt %-99 wt % of at least one of the crystalline polymorphs disclosed herein.

In some embodiments, the pharmaceutical composition comprises 1 wt %-70 wt % of at least one of the crystalline polymorphs disclosed herein.

In some embodiments, the pharmaceutical composition comprises 10 wt %-30 wt % of at least one of the crystalline polymorphs disclosed herein.

The “pharmaceutically acceptable carrier” refers to conventional pharmaceutical carriers suitable for the desired pharmaceutical formulation, for example: a diluent, a vehicle such as water, various organic solvents, etc.; a filler such as starch, sucrose, etc.; a binder such as cellulose derivatives, alginates, gelatin and polyvinylpyrrolidone; a wetting agent such as glycerol; a disintegrating agent such as agar, calcium carbonate and sodium bicarbonate; an absorption enhancer such as quaternary ammoniums; a surfactant such as hexadecanol; an absorption carrier such as Kaolin and soap clay; a lubricant such as talc, calcium stearate, magnesium stearate, polyethylene glycol, etc. In addition, the pharmaceutical composition further comprises at least one other pharmaceutically acceptable excipient such as a decentralized agent, a stabilizer, a thickener, a complexing agent, a buffering agent, a diffusion enhancer, a polymer, a fragrance, a sweetener, and a dye. Preferably, the excipient is suitable for desired formulation and administration type.

In some embodiments, suitable pharmaceutical carriers are chosen from water, various organic solvents and various inert diluents or fillers. If necessary, the pharmaceutical compositions may further comprise one or more additives such as spices, adhesives and excipients. For oral administration, tablets can contain at least one excipient chosen, for example, from citric acid, a variety of disintegrant agents such as starch, alginic acid, and some silicates, and a variety of adhesives such as sucrose, gelatin and Arabic gum. In addition, lubricants including magnesium stearate and talc fillers may, for example, be used in the production of tablets. These components can also, for example, be used to formulate soft and hard gelatin capsules. When an aqueous suspension is needed for oral administration, the active compound may be mixed with at least one component chosen, for example, from a variety of sweeteners and flavoring agents, pigments, and dye combinations. If necessary, a variety of emulsifiers may be employed or suspensions generated; diluents such as water, EtOH, propylene glycol, glycerin, or their combination may also be utilized.

In some embodiments, the pharmaceutical composition further comprises at least one additional active ingredient other than salt of compound 1 or the polymorph form thereof described herein.

The pharmaceutical composition comprising the polymorph(s) of the present invention can be administrated via oral, inhalation, rectal, parenteral or topical administration to a subject who needs treatment. For oral administration, the pharmaceutical composition may be a regular solid formulation such as tablets, pills, coated tablets, powders, granules, capsules and the like, a liquid preparation such as water or oil suspension or other liquid preparation such as syrup, solution, suspension or the like. For parenteral administration, the pharmaceutical composition may be solution, water solution, oil suspension concentrate, lyophilized powder or the like. As a non-limiting example, the formulation of the pharmaceutical composition disclosed herein is selected from tablet, coated tablet, capsule, suppository, nasal spray, and injection. In some embodiments, the formulation of the pharmaceutical composition disclosed herein is chosen from tablets and capsules.

In some embodiments, the pharmaceutical composition may be suitable for oral administration.

In some embodiments, the pharmaceutical compositions disclosed herein may be administered orally in forms such as tablets, capsules, pills, powders, sustained release forms, solutions and/or suspensions; by non-intestinal injection in such form as a sterile solution, suspension or emulsion; through a local treatment form such as paste, cream, or ointment; or via a rectal form such as suppositories. The pharmaceutical compositions disclosed herein may be in a unit dosage form that is suitable for precise dosing applications.

In some embodiments, the pharmaceutical composition is in the form of tablets or capsules.

In some embodiments, the pharmaceutical composition preferably contains 0.05-5000 mg at least one polymorph form of the compound of Formula I disclosed herein. For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

In some embodiments, the pharmaceutical composition of the present invention can be produced by known conventional methods in the pharmaceutical field. For example, one can mix the active ingredient with one or more excipients, and make the mixture into the target formulation.

In another aspect, the present invention provides a use of the salt, polymorph form, and/or pharmaceutical composition of Compound 1 in the manufacturing of a medicament. In some embodiments, the present invention provides a use of the polymorph form of the compound of Formula I disclosed herein, and/or the pharmaceutical composition thereof in the manufacturing of a medicament.

In some embodiments, a medicament thus prepared can be used for the treatment or prevention of cancer or cancer metastasis.

In some embodiments, a medicament thus prepared can be used for the treatment of cancer. In some embodiments, a medicament thus prepared can be used as an Aurora A selective inhibitor.

In some embodiments, the cancer is selected from the group consisting of small cell lung cancer, colorectal cancer, gastric cancer, prostate cancer, breast cancer, triple-negative breast cancer, cervical cancer, head and neck cancer, esophageal cancer, ovarian cancer, thyroid cancer, non-small cell lung cancer, neuroblastoma, and non-Hodgkin lymphoma. Preferred cancers are selected from small cell lung cancer, prostate cancer, triple-negative breast cancer, cervical cancer, neuroblastoma, and head and neck cancer.

In another aspect, the present invention provides a use of the salt, polymorph form, and/or pharmaceutical composition of Compound 1 in therapy. In some embodiments, the present invention provides the polymorph form of the compound of Formula I disclosed herein and/or the pharmaceutical composition thereof for use in therapy.

In another aspect, the present invention provides a use of the salt, polymorph form, and/or pharmaceutical composition of Compound 1 in the treatment or prevention of cancer or cancer metastasis. In some embodiments, the present invention provides the polymorph form of the compound of Formula I disclosed herein and/or the pharmaceutical composition thereof for use in the treatment or prevention of cancer or cancer metastasis.

Furthermore, the present invention provides the polymorph form of the compound of Formula I disclosed herein or the pharmaceutical composition thereof for use in the treatment or prevention of cancer.

In some embodiments, the cancer is selected from the group consisting of small cell lung cancer, colorectal cancer, gastric cancer, prostate cancer, breast cancer, triple-negative breast cancer, cervical cancer, head and neck cancer, esophageal cancer, ovarian cancer, thyroid cancer, non-small cell lung cancer, neuroblastoma and non-Hodgkin lymphoma. Preferred cancers are selected from small cell lung cancer, prostate cancer, triple-negative breast cancer, cervical cancer, neuroblastoma and head and neck cancer.

In another aspect, the present invention provides a method for treating a patient having a condition which is mediated by the activity of Aurora A, comprising administering to the patient a therapeutically effective amount of the salt, polymorph form, and/or pharmaceutical composition of Compound 1. In some embodiments, the present invention provides a method for treating a patient having a condition which is mediated by the activity of Aurora A, comprising administering to the patient a therapeutically effective amount of at least one polymorph form of the compound of Formula I described herein and/or the pharmaceutical composition thereof.

In some embodiments, the condition mediated by the activity of Aurora A is cancer.

In some embodiments, the condition mediated by the activity of Aurora A is small cell lung cancer, colorectal cancer, gastric cancer, prostate cancer, breast cancer, triple-negative breast cancer, cervical cancer, head and neck cancer, esophageal cancer, ovarian cancer, thyroid cancer, non-small cell lung cancer, neuroblastoma, non-Hodgkin lymphoma, or any of combination thereof. Preferred cancers are selected from small cell lung cancer, prostate cancer, triple-negative breast cancer, cervical cancer, neuroblastoma and head and neck cancer.

In still another aspect, at least one of the salts, polymorph forms, and/or pharmaceutical compositions of Compound 1 can be used as an Aurora A selected inhibitor. In some embodiments, at least one polymorph form of the compound of Formula I described herein and/or the pharmaceutical composition thereof can be used as an Aurora A selected inhibitor.

In still another aspect, at least one of the salts, polymorph forms, and/or pharmaceutical compositions of Compound 1 can be used as a medicament. In some embodiments, at least one polymorph form of the compound of Formula I described herein and/or the pharmaceutical composition thereof for use as a medicament.

In still another aspect, the present invention provides a method for treating cancer in a mammal comprising administering to a patient with the disease with a therapeutically effective amount of at least one polymorph form of the compound of Formula I disclosed herein and/or the pharmaceutical composition thereof, wherein the cancer is selected from the group consisting of small cell lung cancer, colorectal cancer, gastric cancer, prostate cancer, breast cancer, triple-negative breast cancer, cervical cancer, head and neck cancer, esophageal cancer, ovarian cancer, thyroid cancer, non-small cell lung cancer, neuroblastoma and non-Hodgkin lymphoma. Preferred cancers are small cell lung cancer, prostate cancer, triple-negative breast cancer, cervical cancer, neuroblastoma and head and neck cancer.

The invention also provides a compound-linker construction, wherein the compound is an Aurora A inhibitor, such as compound 1 or a pharmaceutically acceptable salt thereof that are useful as modulators of Aurora A.

In some embodiments, the compound-linker construction binds to E3 ubiquitin ligand. In some embodiments, the linker maybe absent.

In some embodiments, the compound-linker construction conjugates with a targeting moiety e.g. an antibody, an antibody fragment, a protein, a peptide, or a peptide mimic etc.

In some embodiments, the linker may be any suitable linker disclosed in previous literature or patent applications/patents. In some embodiments, the linker is selected from a chemical linker group, for example four to twenty atoms in shortest length. In some embodiments, the linker comprises one or more cleavage elements, and each cleavage element is independently selected from a self-immolative spacer and a group that is susceptible to cleavage.

The invention further provides a method of treating a disease or condition related to Aurora A protein, comprises administering to a human in need thereof a therapeutically effective amount of the pharmaceutical composition comprising the compound-linker construction of the present invention.

In some embodiments of the method or use, the disease or condition related to Aurora A protein is caner, preferably is small cell lung cancer, colorectal cancer, gastric cancer, prostate cancer, breast cancer, triple-negative breast cancer, cervical cancer, head and neck cancer, esophageal cancer, ovarian cancer, thyroid cancer, non-small cell lung cancer, neuroblastoma and non-Hodgkin lymphoma.

Unless otherwise specified, “treating” as in “treating a disease” in this description means to cause recovery in or to alleviate or suppress a “disease” or one or more “diseases”.

In some embodiments, the methods set forth above may be applied in combination with any chemical therapy, biological therapy, and/or radiation therapy.

In some embodiments, at least 85% of the compound of Formula I present in the pharmaceutical composition is in a crystalline form. As a non-limiting example, at least 85% of the compound of Formula I present in the pharmaceutical composition is at least one chosen from the polymorphs of the compound of Formula I disclosed herein.

In some embodiments, at least 95% of the compound of Formula I present in the pharmaceutical composition is in a crystalline form. As a non-limiting example, at least 95% of the compound of Formula I present in the pharmaceutical composition is at least one chosen from the polymorphs of the compound of Formula I disclosed herein.

In some embodiments, at least 99% of the compound of Formula I present in the pharmaceutical composition is in a crystalline form. As a non-limiting example, at least 99% of the compound of Formula I present in the pharmaceutical composition is at least one chosen from the polymorphs of the compound of Formula I disclosed herein.

New polymorphic, hydrate or solvate forms can provide various advantages, including improved physical characteristics such as stability or solubility. The polymorph forms disclosed herein are purer and more efficacious.

The main peaks described in the crystalline polymorphs above are reproducible and are within the error limit (the specified value±0.2).

In the present invention, “the X-ray powder diffraction pattern shown as in FIG. 1” refers to the X-ray powder diffraction pattern that show major peaks as in FIG. 1, wherein major peaks refer to those with the relative intensity greater than 10%, preferably greater than 30%, relative to the highest peak (with its relative intensity designated to be 100%) in FIG. 1. Likewise, in the present invention, the X-ray powder diffraction pattern shown as in FIG. 3, 5 or 6, refers to the X-ray powder diffraction pattern that show major peaks as in FIG. 3, 5, or 6, wherein major peaks refer to those with the relative intensity greater than 10%, preferably greater than 30%, relative to the highest peak (with its relative intensity designated to be 100%) in FIG. 3, 5, or 6, respectively.

As used herein, the term “a” or “an” as used herein includes the singular and the plural, unless specifically stated otherwise. Therefore, the terms “a,” “an,” or “at least one” can be used interchangeably in this application.

Throughout the application, descriptions of various embodiments use the term “comprising”; however, it will be understood by one of skill in the art, that in some specific instances, an embodiment can alternatively be described using the language “consisting essentially of” or “consisting of.”

As used herein, unless otherwise defined, the term “about” means 10% above or below the value recited. With respect to temperature, unless otherwise defined, the term “about” means the value recited plus or minus 5 degrees.

As used herein, the term “amorphous” refers to a disordered solid state, which may appear during manufacture of the drug substance (crystallization step, drying, and milling) or the drug product (granulation, compression). The X-ray powder diffraction pattern of an amorphous solid exhibits no sharp peaks.

As used herein, the term “solvate” as used herein, means having on a surface, in a lattice or on a surface and in a lattice, a stoichiometric or non-stoichiometric amount of a solvent such as water, acetic acid, ethanol, etc., or mixtures thereof, bound by non-covalent intermolecular forces. The term “hydrate” may be used specifically to describe a solvate comprising water.

As used herein, the term “anhydrous” as used herein, means a crystalline form containing less than about 1% (w/w) of adsorbed moisture as determined by standard methods, such as a Karl Fisher analysis.

In view of the above description and the examples below, one of ordinary skill in the art will be able to practice the invention as claimed without undue experimentation. The foregoing will be better understood with reference to the following examples that detail certain procedures for the preparation of molecules according to the present invention. All references made to these examples are for the purposes of illustration. The following examples should not be considered exhaustive, but merely illustrative of only a few of the many aspects and embodiments contemplated by the present disclosure.

### BRIEF DESCRIPTION OF DRAWINGS

FIG. 1: the X-ray powder diffraction pattern of polymorph form I of the compound of Formula I.

FIG. 2: the DSC analysis spectrum of polymorph form I of the compound of Formula I.

FIG. 3: the X-ray powder diffraction pattern of polymorph form II of the compound of Formula I.

FIG. 4: the DSC analysis spectrum of polymorph form II of the compound of Formula I.

FIG. 5: the X-ray powder diffraction pattern of polymorph form III of the compound of Formula I.

FIG. 6: the X-ray powder diffraction pattern of amorphous of the compound of Formula I.

### DESCRIPTION OF THE EMBODIMENTS

The following examples illustrate the practice of the present subject matter in some of its embodiments, but should not be construed as limiting the scope of the present subject matter. Other embodiments will be apparent to one skilled in the art from consideration of the specification and examples. It is intended that the specification, including the examples, is considered exemplary only, without limiting the scope and spirit of the present subject matter.

### Instrument

### X-Ray Powder Diffraction (XRPD)

The X-ray powder diffraction (XRPD) patterns for the samples were generated on a Bruker D8 Advance X-ray powder diffraction instrument with the Lynxeye detector by Bragg-Brentano method (X-ray source: 40 Kv, 40 mA, Wavelength: 1.54 Å (CuK alpha)). The Scanning range was from 3°-40° 2θ/3°-30° 2θ with the scanning step of 0.02.

Differential Scanning calorimetry (DSC)

Differential scanning calorimetry (DSC) measurement was performed using a Q200 DSC within a range of 0 to 300/320° C., at a heating rate of 10° C./min. The weight of the samples ranged from 0.5-5 mg, the protective gas was N2, and the flow rate of N2 was 50 mL/min.

### Abbreviations Used

RT: room temperature (10-30° C.), >30% RH
THF: tetrahydrofuran
ACN: acetonitrile
MeOH: methanol
EtOH: ethyl alcohol
TFE: trifluoroethanol
DCM: dichloromethane
DIEA: N,N-Diisopropylethylamine
LDA: Lithium diisopropylamide
Pd₂(dba)₃: Tris(dibenzylideneacetone) dipalladium (0)
Xantphos: Dimethylbisdiphenylphosphinoxanthene
DMAP: 4-Dimethylaminopyridine
TFA: 2,2,2-Trifluoroacetic acid
min: minute

### EXAMPLE

### Example 1. Preparation of the Polymorph Form I of the Compound of Formula I

### Method 1

### Step 1: methyl 1-(4-methoxybenzyl)-2-methylpiperidine-4-carboxylate

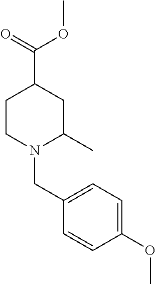 [SMILES: COC(=O)C1CCN(Cc2ccc(OC)cc2)C(C)C1]

A solution of methyl 2-methylpiperidine-4-carboxylate hydrochloride (99.62 g, 514.379 mmol), potassium carbonate (284.360 g, 2.058 mol) in ACN (1.2 L) was refluxed for 2 h. The reaction was cooled to room temperature. The resulting solution was added 4-methoxybenzyl chloride (80.556 g, 514.379 mmol) dropwise and stirred for overnight. After completion, the mixture was filtered and the filtrate was removed under reduced pressure. The residue was dissolved in EtOAc and added 4N HCl aqueous solution to adjust pH to 1. The organic layer was separated, washed with saturated sodium bicarbonate aqueous solution and brine, dried over anhydrous sodium sulfate, filtered and concentrated under reduced pressure. The resulting residue was purified by C18 reversed phase column chromatography eluting with H₂O/ACN to afford methyl 1-(4-methoxybenzyl)-2-methylpiperidine-4-carboxylate (113.09 g) as a yellow oil. LCMS (ESI, m/z): 278 [M+H]⁺.

### Step 2: methyl (2R,4R)-1-(4-methoxybenzyl)-2-methylpiperidine-4-carboxylate

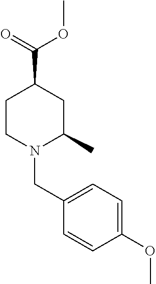 [SMILES: COC(=O)[C@@H]1CCN(Cc2ccc(OC)cc2)[C@H](C)C1]

Two enantiomers of methyl 1-(4-methoxybenzyl)-2-methylpiperidine-4-carboxylate (112.5 g) was separated using chiral chromatography. Stationary phase: CHIRALPAK IA, Column size: 0.46 cm I.D.×15 cm L, mobile phase: n-hexane/EtOH 0.1% DIEA=75/25 (V/V), flow rate: 1.0 mL/min, wave length: UV 210 nm, temperature: 25° C. The first eluted enantiomer was collected as the title compound (52.05 g, 46.3% yield; Rr=2.615 min; LCMS (ESI, m/z): 278 [M+H]⁺), the second eluted enantiomer was collected to give 48.12 g, Rr=4.449 min; LCMS (ESI, m/z): 278 [M+H]⁺.

### Step 3: methyl-(2R,4R)-2-methylpiperidine-4-carboxylate-hydrochloride

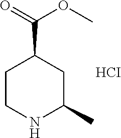

A solution of methyl (2R,4R)-1-(4-methoxy benzyl)-2-methylpiperidine-4-carboxylate (50.75 g, 182.977 mmol) in methanol (500 mL) was added Pd/C (10.44 g), purged with nitrogen and pressurized with H₂. The mixture was heated to 45° C. and stirred for 16 h. After completion, the resulting mixture was filtered and the filter cake was washed with methanol (200 mL×3). The filtrate was collected and removed under reduced pressure. The resulting residue was added 4M HCl/ethyl acetate and stirred 2 h at room temperature. The solid was collected. The filter cake was rinsed with ethyl acetate and dried under vacuum to afford methyl-(2R,4R)-2-methyl piperidine-4-carboxylate hydrochloride (32.97 g, Y=93%) as a white solid. LCMS (ESI, m/z): 158 [M+H]⁺.

### Step 4:1-(tert-butyl)-4-methyl (2R,4R)-2-methylpiperidine-1,4-dicarboxylate

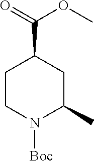 [SMILES: COC(=O)[C@@H]1CCN(C(=O)OC(C)(C)C)[C@H](C)C1]

A solution of methyl-(2R,4R)-2-methylpiperidine-4-carboxylate hydrochloride (32.87 g, 169.721 mmol), N,N-diisopropylethylamine (102.58 g, 793.702 mmol), N-(4-pyridyl) dimethylamine (3.14 g, 25.703 mmol) and di-tert-butyl dicarbonate (56.31 g, 258.011 mmol) in DCM (500 mL) was stirred at room temperature for 2 h. The resulting solution was diluted with water and extracted with DCM. The organic layer was dried over anhydrous sodium sulfate, filtered and concentrated under reduced pressure. The resulting residue was purified by a silica gel column chromatography (eluent: 0˜100% hexane/DCM) to afford 1-(tert-butyl) 4-methyl (2R,4R)-2-methylpiperidine-1,4-dicarboxylate (37.11 g, Y=84.97%) as a yellow oil. LCMS (ESI, m/z): 258 [M+H]⁺.

### Step 5: (2R,4R)-1-(tert-butoxycarbonyl)-2-methylpiperidine-4-carboxylic acid

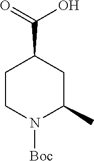 [SMILES: C[C@@H]1C[C@H](C(=O)O)CCN1C(=O)OC(C)(C)C]

A mixture of 1-(tert-butyl) 4-methyl (2R,4R)-2-methylpiperidine-1,4-dicarboxylate (37.11 g, 144.214 mmol) in THF (260 mL) and water (130 mL) was added lithium hydroxide (16.95 g, 707.775 mmol). The reaction mixture was stirred at room temperature for overnight. The resulting solution was added IN HCl aqueous solution to adjust pH to 3˜4 and extracted with ethyl acetate. The organic layer was washed with brine, dried over anhydrous sodium sulfate, and concentrated under reduced pressure to give (2R,4R)-1-(tert-butoxycarbonyl)-2-methyl piperidine-4-carboxylic acid (39.5 g, crude) as a yellow oil. LCMS (ESI, m/z): 244 [M+H]⁺.

### Step 6: di-tert-butyl-(2R,4R)-2-methylpiperidine-1,4-dicarboxylate

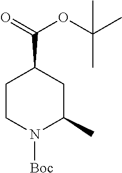 [SMILES: C[C@@H]1C[C@H](C(=O)OC(C)(C)C)CCN1C(=O)OC(C)(C)C]

A solution of (2R, 4R)-1-(tert-butoxycarbonyl)-2-methylpiperidine-4-carboxylic acid (39.5 g, crude), N-(4-pyridyl)-dimethylamine (4.84 g, 39.618 mmol) and di-tert-butyl-di-carbonate (63.94 g, 292.972 mmol) in tert-Butanol (400 mL) was stirred at room temperature for overnight under nitrogen. After completion, the resulting solution was diluted with water and extracted with ethyl acetate. The organic layer was washed with brine, dried over anhydrous sodium sulfate, and concentrated under reduced pressure. The resulting residue by a silica gel column chromatography (eluent: 0˜10% ethyl acetate/hexane) to afford di-tert-butyl (2R,4R)-2-methylpiperidine-1,4-di-carboxylate (35.7 g, Y=73%) as a white solid. LCMS (ESI, m/z): 300 [M+H]⁺.

### Step 7: di-tert-butyl (2R,4R)-4-((6-chloro-5-fluoropyridin-2-yl)methyl)-2-methylpiperidine-1,4-dicarboxylate

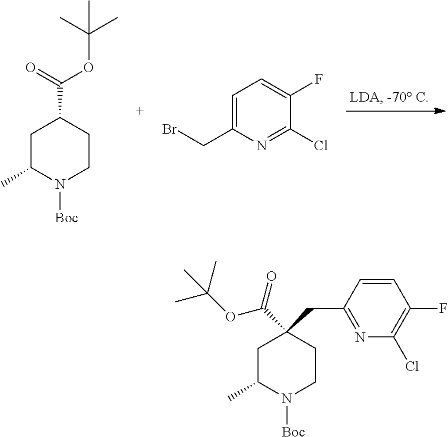 [SMILES: C[C@@H]1C[C@H](C(=O)OC(C)(C)C)CCN1C(=O)OC(C)(C)C ; Fc1ccc(CBr)nc1Cl ; C[C@@H]1C[C@](Cc2ccc(F)c(Cl)n2)(C(=O)OC(C)(C)C)CCN1C(=O)OC(C)(C)C]

A solution of diisopropylamine (20.42 g, 201.76 mmol) in THF (44 ml) was cooled to −70˜-80° C. under nitrogen. n-Butyl lithium 2.5 M in THF (80 mL, 200 mmol) was added and the resulting solution was stirred at 0° C. for 30 min. Then a solution of di-tert-butyl (2R,4R)-2-methylpiperidine-1,4-di-carboxylate (28.16 g, 94.05 mmol) in THF (88 ml) was added slowly and the reaction was stirred at −50° C.˜−70° C. for 1 h.

A solution of 2-(bromomethyl)-6-chloro-3-fluoropyridine (24.2 g, 107.23 mmol) was added to above solution and the reaction solution was stirred at −70° C.˜−80° C. for 2 h. After completion, the resulting solution was quenched with saturated ammonium chloride aqueous solution (1.1 L) and extracted with EtOAc (2.2 L×3). The organic layer was concentrated under reduced pressure. The resulting residue was purified by C18 reverse phase chromatography eluting with H₂O/ACN to afford 19.04 g of the title compound. LCMS (ESI, m/z): 443 [M+H]⁺.

### Step 8: di-tert-butyl (2R,4R)-4-((6-chloro-5-fluoro-4-methylpyridin-2-yl)methyl)-2-methylpiperidine-1,4-dicarboxylate

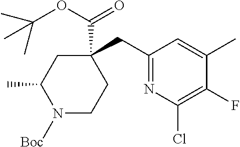 [SMILES: Cc1cc(C[C@@]2(C(=O)OC(C)(C)C)CCN(C(=O)OC(C)(C)C)[C@H](C)C2)nc(Cl)c1F]

A solution of diisopropylamine (9.84 g, 97.24 mmol) in THF (40 ml) was added n-butyl lithium 2.5 M in THF (33.6 ml, 84 mmol) at −60˜−70° C. under nitrogen. The solution was stirred for 1 h at −10° C.˜0° C. The resulting solution was slowly added di-tert-butyl (2R,4R)-4-((6-chloro-5-fluoropyridin-2-yl)methyl)-2-methylpiperidine-1,4-dicarboxylate (18.68 g, 42.17 mmol) in THF (40 ml). The solution was stirred at −60° C.˜−70° C. for 1 h.

A solution of iodomethane (7.28 g, 51.29 mmol) in THF (8 ml) was added to the above solution. The resulting solution was stirred at −60° C.˜−70° C. for 2 h. After completion, the reaction was quenched with saturated ammonium chloride aqueous solution and extracted with EtOAc. The organic layer was combined, dried over anhydrous sodium sulfate and concentrated under reduced pressure. The residue was purified by C18 reverse phase chromatography eluting with H₂O/ACN to afford 11.36 g of the title compound as a yellow oil. LCMS (ESI, m/z): 457 [M+H]⁺.

### Step 9: di-tert-butyl-(2R,4R)-4-((6-((1-(tert-butyl)-5-methyl-1H-pyrazol-3-yl)amino)-5-fluoro-4-methylpyridin-2-yl)methyl)-2-methylpiperidine-1,4-dicarboxylate

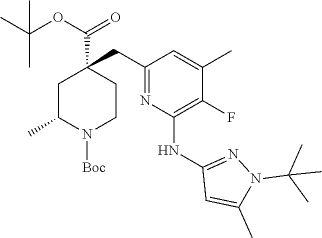 [SMILES: Cc1cc(C[C@@]2(C(=O)OC(C)(C)C)CCN(C(=O)OC(C)(C)C)[C@H](C)C2)nc(Nc2cc(C)n(C(C)(C)C)n2)c1F]

A mixture of di-tert-butyl-(2R,4R)-4-((6-chloro-5-fluoro-4-methylpyridin-2-yl)methyl)-2-methylpiperidine-1,4-dicarboxylate (11.10 g, 24.25 mmol), tris(dibenzylideneacetone) dipalladium (6.52 g, 7.12 mmol), dimethylbisdiphenylphosphinoxanthene (4.24 g, 7.33 mmol), 1-tert-butyl-3-methyl-1H-pyrazol-5-amine (4.88 g, 31.84 mmol) and K₃PO₄ (22.24 g, 104.78 mmol) in 1,4-dioxane (120 ml) was stirred at 110° C. for 5 h under nitrogen. The resulting solution was cooled to room temperature, diluted with brine and extracted with ethyl acetate. The organic layer was dried over anhydrous sodium sulfate, filtered and concentrated under reduced pressure. The residue was purified by C18 reverse phase chromatography eluting with H₂O/ACN to afford (11.72 g, 20.356 mmol, Y=83%) of the title compound as a yellow solid. LCMS (ESI, m/z): 574 [M+H]⁺.

### Step 10: tert-butyl-(2R,4R)-4-((6-((1-(tert-butyl)-5-methyl-1H-pyrazol-3-yl)amino)-5-fluoro-4-methylpyridin-2-yl)methyl)-2-methylpiperidine-4-carboxylate

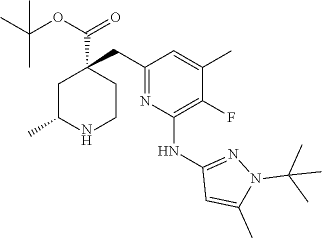 [SMILES: Cc1cc(C[C@@]2(C(=O)OC(C)(C)C)CCN[C@H](C)C2)nc(Nc2cc(C)n(C(C)(C)C)n2)c1F]

A solution of di-tert-butyl-(2R,4R)-4-((6-((1-(tert-butyl)-5-methyl-1H-pyrazol-3-yl)amino)-5-fluoro-4-methylpyridin-2-yl)methyl)-2-methylpiperidine-1,4-dicarboxylate (11.68 g, 20.356 mmol) in dichloromethane (120 ml) was added trifluoroacetic acid (12 ml) and stirred at room temperature for 4 h. After completion, the reaction was quenched with saturated sodium bicarbonate aqueous solution (400 ml) and extracted with DCM. The organic layer was dried over anhydrous sodium sulfate, filtered and concentrated under reduced pressure. The crude product was purified by C18 reverse phase column chromatography eluting with H₂O/ACN/0.03 formic acid to afford (6.80 g, 14.36 mmol, Y=71%) of the title compound as a yellow solid. LCMS (ESI, m/z): 474 [M+H]⁺.

### Step 11: tert-butyl-(2R,4R)-4-((6-((1-(tert-butyl)-5-methyl-1H-pyrazol-3-yl)amino)-5-fluoro-4-methylpyridin-2-yl)methyl)-1-(3-chloro-2-fluorobenzyl)-2-methylpiperidine-4-carboxylate

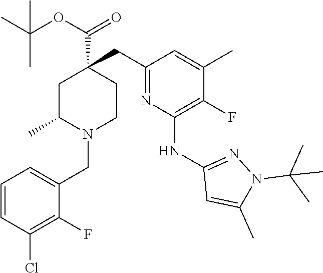 [SMILES: Cc1cc(C[C@@]2(C(=O)OC(C)(C)C)CCN(Cc3cccc(Cl)c3F)[C@H](C)C2)nc(Nc2cc(C)n(C(C)(C)C)n2)c1F]

A mixture of tert-butyl (2R,4R)-4-((6-((1-(tert-butyl)-5-methyl-1H-pyrazol-3-yl)amino)-5-fluoro-4-methylpyridin-2-yl)methyl)-2-methylpiperidine-4-carboxylate (6.80 g, 14.36 mmol) and potassium carbonate (10.16 g, 73.55 mmol), 1-(bromomethyl)-3-chloro-2-fluorobenzene (3.64 g, 16.29 mmol) in ACN (80 mL) was stirred for 6 h at room temperature. After completion, the resulting mixture was filtered and concentrated under reduced pressure. The crude product was purified by silica gel column chromatography eluting with EtOAc/n-hexane (0˜30%) to afford (7.84 g, 15.27 mmol, Y=89%) of the title compound as a yellow solid. LCMS (ESI, m/z): 616 [M+H]⁺.

### Step 12: (2R,4R)-1-(3-chloro-2-fluorobenzyl)-4-((5-fluoro-4-methyl-6-((5-methyl-1H-pyrazol-3-yl)amino) pyridin-2-yl)methyl)-2-methylpiperidine-4-carboxylic acid

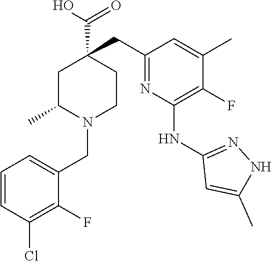 [SMILES: Cc1cc(Nc2nc(C[C@@]3(C(=O)O)CCN(Cc4cccc(Cl)c4F)[C@H](C)C3)cc(C)c2F)n[nH]1]

A solution of tert-butyl (2R,4R)-4-((6-((1-(tert-butyl)-5-methyl-1H-pyrazol-3-yl)amino)-5-fluoro-4-methylpyridin-2-yl)methyl)-1-(3-chloro-2-fluorobenzyl)-2-methylpiperidine-4-carboxylate (7.61 g, 12.35 mmol) in formic acid (60 mL) was stirred at reflux for 1.5 h. After completion, the resulting solution was concentrated. The residue was dissolved in water (40 mL) at 0° C., and adjusted pH-6˜7 with sodium hydroxide aqueous solution (5 M). The resulting mixture was extracted with DCM. The organic layer was dried over anhydrous sodium sulfate, filtered and concentrated under reduced pressure. The crude product was purified by C18 reverse phase column chromatography eluting with MeOH/water to afford (6.22 g, 11.453 mmol, Y=93%) of the title compound as a white solid. LCMS (ESI, m/z): 504 [M+H]⁺. ¹H NMR (400 MHZ, MeOD) δ 7.47-7.33 (m, 2H), 7.12 (t, 1H), 6.47 (d, J=4.5 Hz, 1H), 5.93 (s, 1H), 4.29 (d, J=13.6 Hz, 1H), 3.68 (d, J=13.5 Hz, 1H), 3.13 (s, 1H), 3.04 (d, J=13.4 Hz, 1H), 2.96 (d, J=13.5 Hz, 1H), 2.87 (d, J=12.3 Hz, 1H), 2.75 (t, 1H), 2.16 (s, 3H), 2.13 (s, 3H), 1.83 (d, J=11.2 Hz, 2H), 1.75 (t, 2H), 1.22 (d, J=6.0 Hz, 3H).

### Step 13: Preparation of Polymorph Form I

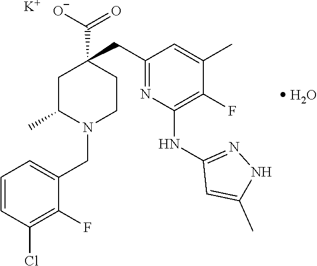 [SMILES: [K+] ; Cc1cc(Nc2nc(C[C@@]3(C(=O)[O-])CCN(Cc4cccc(Cl)c4F)[C@H](C)C3)cc(C)c2F)n[nH]1 ; "· H2O"]

(2R,4R)-1-(3-chloro-2-fluorobenzyl)-4-((5-fluoro-4-methyl-6-((3-methyl-1H-pyrazol-5-yl)amino) pyridin-2-yl)methyl)-2-methylpiperidine-4-carboxylic acid (6.05 g) was dissolved in methanol (25 mL). The mixture was added KOH (0.71 g) in methanol (3 mL) and then stirred for 3 h at room temperature. MBTE (70 mL) was dropwise added to the above mixture at 0˜5° C. within 2 h, then stirred for 5˜6 h. The resulting mixture was filtered and the cake was washed with MBTE (15 mL), dried under vacuum at 45˜55° C. for 6˜8 h to provide potassium (2R,4R)-1-(3-chloro-2-fluorobenzyl)-4-((5-fluoro-4-methyl-6-((3-methyl-1H-pyrazol-5-yl)amino) pyridin-2-yl)methyl)-2-methylpiperidine-4-carboxylate monohydrate (5.45 g).

¹H NMR (400 MHZ, DMSO) δ 13.02 (s, 1H), 9.50 (s, 1H), 7.46-7.40 (m, 2H), 7.21-7.17 (t, 1H), 6.48 (s, 1H), 5.66 (s, 1H), 4.01-4.05 (d, J=16.0 Hz, 1H), 3.22-3.26 (d, J=16 Hz, 1H), 3.01-2.98 (d, J=12 Hz, 1H), 2.91-2.88 (d, J=12 Hz, 1H), 2.56 (s, 1H), 2.46 (s, 1H), 2.30-2.24 (t, 1H), 2.13 (s, 3H), 2.11 (s, 3H), 1.71-1.66 (t, 1H), 1.56-1.45 (m, 2H), 1.32-1.30 (t, J=6 Hz, 1H), 1.04-1.03 (d, J=4 Hz, 3H).

### Method 2

About 30 mg of amorphous of compound of Formula I obtained from Example 2 was dissolved in a solvent to obtain a suspension. The suspension was stirred at a corresponding temperature for 12 h. The suspension was centrifuged to separate. The resulting solid was dried overnight under vacuum and identified by XRPD as polymorph form I. The specific preparation parameters are shown in Table 1.

TABLE 1

| Temp. | | | Slurry |
|---|---|---|---|
| (° C.) | Solvent 1 | Solvent 1 (mL) | Time (h) |
| RT | Isopropanol | 0.6 | 0.5 |
| RT | Acetone | 0.6 | 0.5 |
| RT | Ethyl acetate | 0.6 | 0.5 |
| RT | Acetonitrile | 0.6 | 0.5 |

### Method 3

At RT, 10 mg of amorphous of compound of Formula I obtained from Example 2 was placed into a centrifuge tube, then the tube was placed in EtOH or DCM atmosphere for 4 days, the solid was analyzed by XRPD, and determined to be polymorph form I.

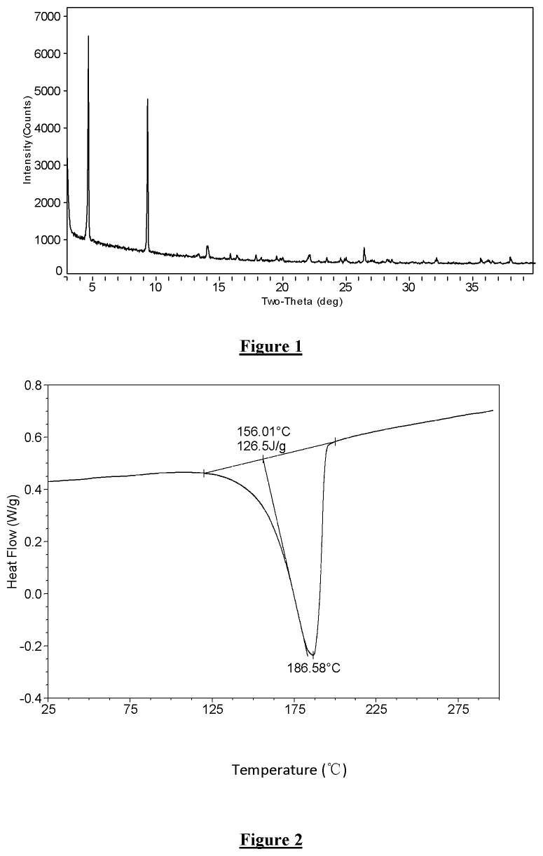

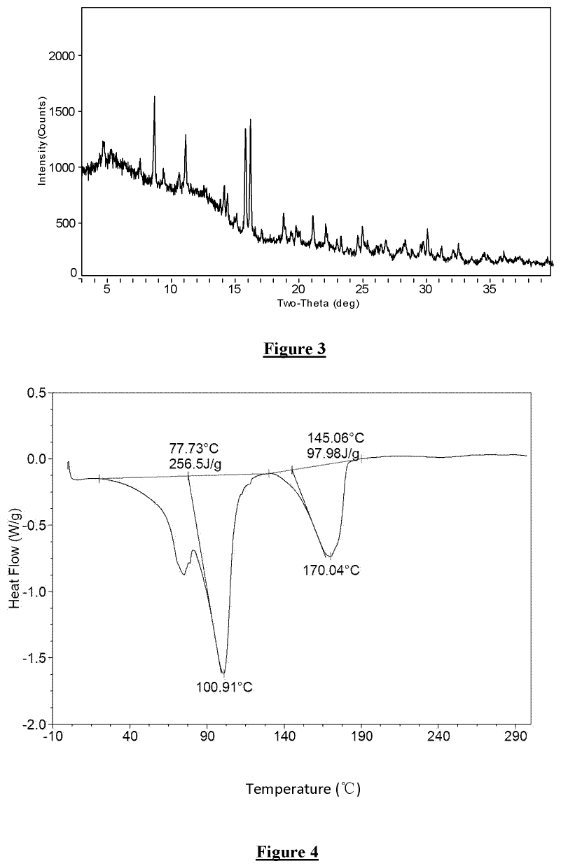

The XRPD of polymorph form I is substantially characterized in FIG. 1. The XRPD data of polymorph form I are summarized below in Table 2.

TABLE 2

| | Relative intensity |
|---|---|
| 2θ angle (°) ± 0.2 | (%) |
| 4.7 | 100 |
| 9.3 | 75.3 |
| 11.7 | 1.3 |
| 13.4 | 1.7 |
| 14.1 | 5.8 |
| 15.9 | 2.9 |
| 16.4 | 2 |
| 17.9 | 2.6 |
| 18.3 | 1.6 |
| 19.5 | 2.6 |
| 19.8 | 1.5 |
| 20.0 | 2 |
| 22.1 | 3.6 |
| 22.8 | 0.9 |
| 23.5 | 2.3 |
| 24.6 | 2.3 |
| 25.0 | 2.5 |
| 26.0 | 0.9 |
| 26.4 | 7.6 |
| 27.1 | 1 |
| 27.9 | 1.1 |
| 28.3 | 1.9 |
| 28.6 | 1.7 |
| 30.3 | 0.8 |
| 30.4 | 0.7 |
| 31.1 | 1.4 |
| 32.2 | 2.9 |
| 33.1 | 0.7 |
| 33.5 | 0.8 |
| 34.8 | 0.8 |
| 35.6 | 2.4 |
| 36.2 | 1.7 |
| 36.6 | 1.3 |
| 37.2 | 0.8 |
| 38.0 | 3.3 |

### Example 2. Preparation of Amorphous

### Method 1

About 20 mg of Polymorph Form I of the compound of formula I was dissolved in 5 ml TFE by heating with sonication to obtain a clear solution. The solution was filtered, and the filtrate was evaporated at RT to obtain the amorphous.

### Method 2

About 30 mg of Polymorph Form I of the compound of formula I was dissolved in 0.5 ml TFE with sonication to obtain a solution. The solution was filtered, and the filtrate was evaporated at RT to obtain the amorphous.

### Method 3

About 100 mg of Polymorph Form I of the compound of formula I was dissolved in 3 mL methanol, 0.6 mL isopropyl ether and 0.6 mL acetonitrile with sonication. The solution was filtered and rotary evaporated at RT to obtain the amorphous.

### Method 4

About 100 mg of Polymorph Form I of the compound of formula I was dissolved in 3 mL methanol, 0.6 mL isopropyl ether and 0.6 mL acetonitrile to dissolve the sample with sonication. The solution was filtered, then the allochroic silicagel was added. The filtrate was rotary evaporated at RT to obtain the amorphous.

### Example 3. Preparation of Polymorph Form II

### Method 1

About 20 mg of Polymorph Form I of the compound of formula I was dissolved in Acetone (2.0 ml) and water (0.8 ml) by heating with sonication to obtain a clear solution. The solution was filtered, and the filtrate was evaporated at RT to obtain the Polymorph Form II of the compound of Formula I.

### Method 2

About 20 mg of Polymorph Form I of the compound of formula I was dissolved in THF (2.0 ml) and water (0.6 ml) by heating with sonication to obtain a clear solution. The solution was filtered, and the filtrate was evaporated at RT to obtain the Polymorph Form II of the compound of Formula I.

### Method 3

About 30 mg of Polymorph Form I of the compound of formula I was dissolved in THF (0.5 ml) and water (0.2 ml) by heating with sonication to obtain a clear solution. The solution was filtered, and the filtrate was evaporated at RT to obtain the Polymorph Form II of the compound of Formula I.

### Method 4

At RT, 10 mg of the amorphous form of compound of Formula I obtained from Example 2 was placed into a centrifuge tube, then the tube was placed in water atmosphere for 4 days, the solid was analyzed by XRPD, and determined to be polymorph form II of the compound of Formula I.

### Method 5

About 100 mg of Polymorph Form I of the compound of formula I was dissolved in 4.0 ml acetone and 1.6 ml water to obtain a clear solution at 40° C. The solution was filtered and evaporated at RT to obtain the Polymorph Form II of the compound of Formula I.

The XRPD of polymorph form II is substantially characterized in FIG. 3. The XRPD data of polymorph form II are summarized below in Table 3.

TABLE 3

| | Relative intensity |
|---|---|
| 2θ angle (°) ± 0.2 | (%) |
| 4.4 | 9.8 |
| 4.7 | 19 |
| 5.3 | 10.6 |
| 8.7 | 74 |
| 9.4 | 16.1 |
| 10.6 | 13.6 |
| 11.1 | 49.9 |
| 12.5 | 8.8 |
| 13.8 | 8.7 |
| 14.1 | 23.1 |
| 15.1 | 12.6 |
| 15.8 | 90.6 |
| 16.2 | 100 |
| 17.1 | 8.8 |
| 18.8 | 23.8 |
| 19.4 | 8.3 |
| 19.8 | 13.8 |
| 20.1 | 9.1 |
| 21.1 | 25.5 |
| 22.1 | 20.7 |
| 23.0 | 9.3 |
| 23.3 | 12.4 |
| 23.8 | 5 |
| 24.6 | 13.2 |
| 25.0 | 23.2 |
| 25.4 | 10.9 |
| 26.1 | 5.4 |
| 26.5 | 9.4 |
| 26.8 | 13.8 |
| 27.7 | 6.9 |
| 28.0 | 8.5 |
| 28.3 | 15.4 |
| 28.9 | 6.3 |
| 29.5 | 11.2 |
| 29.8 | 13.9 |
| 30.1 | 24.9 |
| 30.4 | 7.3 |
| 30.9 | 5.3 |
| 31.2 | 11.5 |
| 32.1 | 9.2 |
| 32.5 | 16.1 |
| 33.5 | 5.2 |
| 34.5 | 9.1 |
| 34.8 | 6.3 |
| 35.8 | 5.3 |
| 36.1 | 9.4 |
| 37.0 | 4.7 |
| 37.3 | 4.5 |
| 39.5 | 6.5 |

### Example 4. Preparation of Polymorph Form III

### Method 1

A proper amount of polymorph form I was heated to 150° C. and kept for 5 min on hot-stage to obtain polymorph form III.

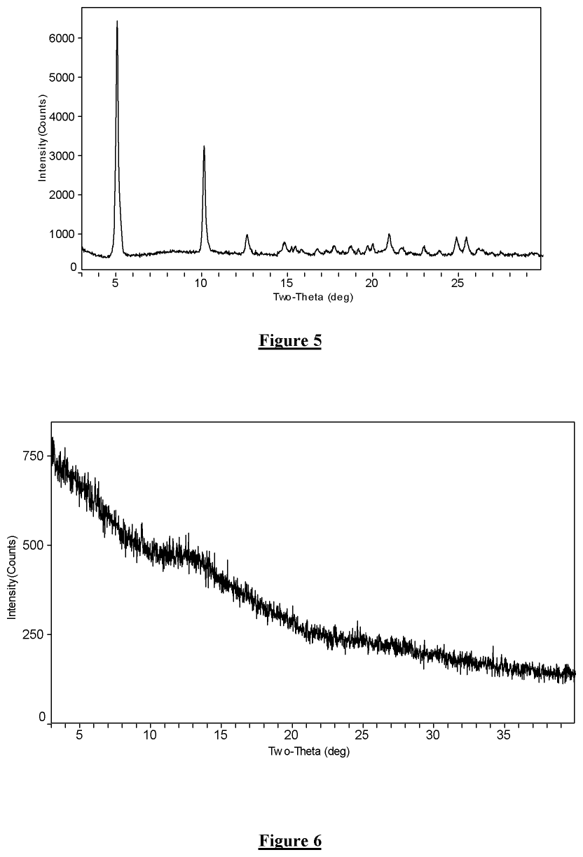

The XRPD of polymorph form III is substantially characterized in FIG. 5. The XRPD data of polymorph form III are summarized below in Table 4.

TABLE 4

| | Relative intensity |
|---|---|
| 2θ angle (°) ± 0.2 | (%) |
| 5.1 | 100 |
| 8.3 | 1.2 |
| 10.1 | 45.4 |
| 12.7 | 8.2 |
| 13.1 | 1.7 |
| 14.8 | 4.7 |
| 15.2 | 3.2 |
| 15.5 | 3.2 |
| 15.8 | 1.2 |
| 16.7 | 2.4 |
| 17.3 | 1.6 |
| 17.7 | 3.4 |
| 18.7 | 3 |
| 19.2 | 1.9 |
| 19.7 | 3.1 |
| 20.0 | 3.8 |
| 21.0 | 7.9 |
| 21.7 | 2.4 |
| 23.0 | 3.9 |
| 23.9 | 2.1 |
| 24.9 | 7.4 |
| 25.5 | 7.3 |
| 26.2 | 2.7 |
| 26.4 | 2.3 |
| 27.5 | 1.8 |
| 29.3 | 1.3 |

### Example 5. Stability Experiment

1. Sample: polymorph form I.

Experimental procedure: about 20 mg samples were weighed and put into a 5 mL glass vial. The crystal forms stability test was carried out under the specified experimental conditions.

Experimental results: polymorph form I remained unchanged under long-term and accelerated conditions for 14 days.

The specific results were shown in the Table 5 below.

TABLE 5

| | Long-time (25° C. ± 2° C., | | | | Accelerated (40° C. ± 2° C., | | | |
|---|---|---|---|---|---|---|---|---|
| | 65% RH ± 10% RH) | | | | 75% RH ± 10% RH) | | | |
| | uncapped, | | | | uncapped, | | | |
| Experimental | protected from | | Sealed, protected | | protected from | | Sealed, protected | |
| Conditions | light | | from light | | light | | from light | |
| Test Time | 7 days | 14 days | 7 days | 14 days | 7 days | 14 days | 7 days | 14 days |

| | | |
|---|---|---|
| Results (XRPD) | Polymorph form I | Polymorph form I |

2. Polymorph form II is converted to polymorph form I after drying under 40° C. overnight.

3. Polymorph form III is converted to polymorph form I when cooled back to room temperature.

4. The amorphous is sealed for more than two weeks and transformed to Polymorph form I.

### Example 6. PK in Rat

The polymorph form I of the compound Formula I was prepared into suspension in 0.5% CMC-Na with ddH₂O, then the SD rats were given the polymorph form I orally.

The plasma samples were collected at 0 hour (pre-dose), 0.083, 0.25, 0.5, 1, 2, 4, 6, 8, 24 hours post-dose. Plasma drug concentration was detected by LC-MS/MS. Pharmacokinetic parameters were calculated using WinNonlin's software with non-compartmental model.

The results are shown in Table A.

TABLE A
PO: 5 mg/kg

| Cₘₐₓ | AUCₗₐₛₜ | t_{1/2} |
|---|---|---|
| (ng/mL) | (h*ng/mL) | (h) |
| 12382 | 93347 | 4.11 |

### Example 7. PK in Dog

The polymorph form I of the compound Formula I was prepared into suspension in 0.5% CMC-Na with ddH₂O, then the Beagle dogs were given the polymorph form I orally. Then plasma samples were collected at 0 hour (pre-dose), 0.083, 0.25, 0.5, 1, 2, 4, 6, 8, 24 hours post-dose. Plasma drug concentration was detected by LC-MS/MS. Pharmacokinetic parameters were calculated using WinNonlin's software with non-compartmental model.

The results are shown in Table B.

TABLE B

| | |
|---|---|
| PO: 3 mg/kg | PO: 10 mg/kg |

| Cₘₐₓ | AUCₗₐₛₜ | t_{1/2} | Cₘₐₓ | AUCₗₐₛₜ | t_{1/2} |
|---|---|---|---|---|---|
| (ng/mL) | (h*ng/mL) | (h) | (ng/mL) | (h*ng/mL) | (h) |
| 14350 | 45547 | 8.12 | 45025 | 164712 | 4.51 |

From the above, the polymorph form I of the compound Formula I according to the invention showed a great absorption and a high exposure in vivo.

Based on the above results, polymorph form I is the most stable anhydrous at room temperature.

Although the present invention has been fully described in connection with embodiments thereof with reference to the accompanying drawings, it is to be noted that various change and modifications will become apparent to those skilled in the art. Such changes and modifications are to be understood as being included within the scope of the present invention as defined by the appended claims.

All publications, patents and patent applications mentioned in this specification are herein incorporated in their entirety by reference into the specification, to the same extent as if each individual publication, patent or patent application was specifically and individually indicated to be incorporated herein by reference.

What is claimed is:

1-35. (canceled)

36. A polymorph form of the compound of Formula I, its hydrate and/or a solvate thereof

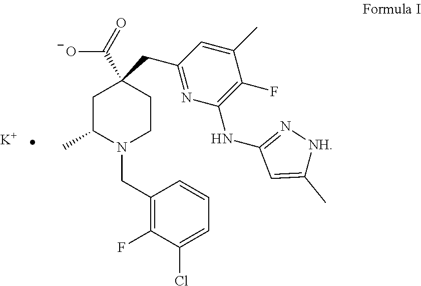

37. The polymorph form of claim 36, wherein the polymorph form is polymorph form I and characterized by the X-ray powder diffraction pattern having peaks at diffraction angles 2θ of 4.7°±0.2°, 9.3°±0.2° and 14.1°±0.2°.

38. The polymorph form of claim 36, wherein the polymorph form is polymorph form I and characterized by the X-ray powder diffraction pattern having peaks at diffraction angles 2θ of 4.7°±0.2°, 9.3°±0.2°, 14.1°±0.2°, 22.1°±0.2° and 26.4°±0.2°.

39. The polymorph form of claim 36, characterized by the X-ray powder diffraction pattern having peaks at diffraction angles 2θ of 4.7°±0.2°, 9.3°±0.2°, 14.1°±0.2°, 15.9°±0.2°, 17.9°±0.2°, 22.1°±0.2°, 26.4°±0.2°, 32.2°±0.2° and 38.0°±0.2°.

40. The polymorph form of claim 36, characterized by the X-ray powder diffraction pattern having peaks at diffraction angles 2θ of 4.7°±0.2°, 9.3°±0.2°, 14.1°±0.2°, 15.9°±0.2°, 17.9°±0.2°, 19.5°±0.2°, 22.1°±0.2°, 23.5°±0.2°, 24.6°±0.2°, 25.0°±0.2°, 26.4°±0.2°, 32.2°±0.2°, 35.6°±0.2° and 38.0°±0.2°.

41. The polymorph form of claim 36, wherein the X-ray powder diffraction pattern is shown as in FIG. 1.

42. The polymorph form of claim 36, wherein the polymorph form is polymorph form II and characterized by the X-ray powder diffraction pattern having peaks at diffraction angles 2θ of 8.7°±0.2°, 11.1°±0.2° and 15.8°±0.2°.

43. The polymorph form of claim 42, wherein the polymorph form is polymorph form II and characterized by the X-ray powder diffraction pattern having peaks at diffraction angles 2θ of 8.7°±0.2°, 11.1°±0.2°, 15.8°±0.2°, 16.2°±0.2° and 21.1°±0.2°.

44. The polymorph form of claim 42, characterized by the X-ray powder diffraction pattern having peaks at diffraction angles 2θ of 8.7°±0.2°, 11.1°±0.2°, 14.1°±0.2°, 15.8°±0.2°, 16.2°±0.2°, 18.8°±0.2°, 21.1°±0.2°, 25.0°±0.2° and 30.1°±0.2°.

45. The polymorph form of claim 42, characterized by the X-ray powder diffraction pattern having peaks at diffraction angles 2θ of 4.7°±0.2°, 8.7°±0.2°, 9.4°±0.2°, 11.1°±0.2°, 14.1°±0.2°, 15.8°±0.2°, 16.2°±0.2°, 18.8°±0.2°, 21.1°±0.2°, 22.1°±0.2°, 25.0°±0.2°, 30.1°±0.2° and 32.5°±0.2°.

46. The polymorph form of claim 42, wherein the X-ray powder diffraction pattern is substantially shown as in FIG. 3.

47. The polymorph form of claim 36, wherein the polymorph form has a purity of ≥95%.

48. The polymorph form of claim 36, wherein the polymorph form has a purity of ≥99%.

49. The polymorph form of claim 36, wherein the polymorph form has a purity of ≥99.5%.

50. A process for preparing a polymorph form of claim 37, comprising:
a) 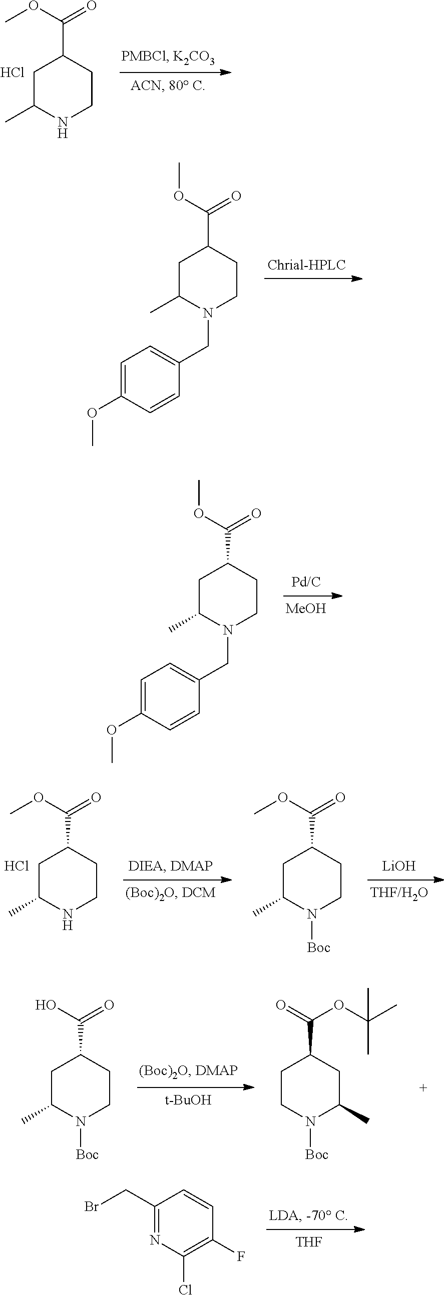 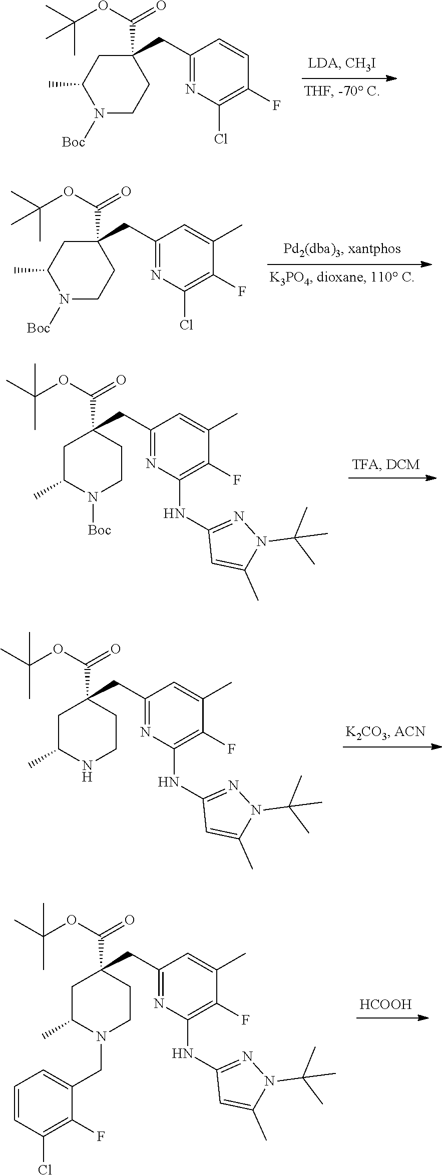 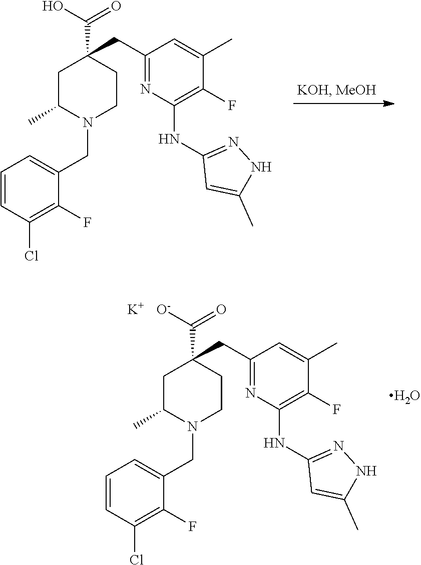
b) placing an amorphous form of the compound of Formula I in ethanol or in dichloromethane atmosphere for 4 days; or
c) drying the polymorph form II of the compound of formula I under 40° C. for overnight, wherein the X-ray powder diffraction pattern of the polymorph form II is substantially shown as in FIG. 3; or
d) adding an amorphous form of the compound of Formula I in a suitable solvent to obtain a suspension, which is stirred, centrifuged, and dried to obtain the polymorph form; wherein the solvent is selected from isopropanol, acetone, ethyl acetate, or acetonitrile;
to obtain the polymorph form of claim 37.

51. A pharmaceutical composition comprising a therapeutically effective amount of the polymorph form of claim 36 and at least one pharmaceutically acceptable carrier.

52. The pharmaceutical composition of claim 51, further comprising at least one additional active ingredient.

53. The pharmaceutical composition of claim 51, wherein the pharmaceutical composition is suitable for oral administration.

54. The pharmaceutical composition of claim 51, wherein the pharmaceutical composition is in a form of tablets or capsules.

55. The pharmaceutical composition of claim 51, wherein the composition comprises 0.01 wt %-99 wt % of the polymorph form.

56. The pharmaceutical composition of claim 55 wherein the composition comprises 1 wt %-70 wt % of the polymorph form.

57. The pharmaceutical composition of claim 56, wherein the composition comprises 10 wt %-30 wt % of the polymorph form.

58. A method for treating a patient having a condition mediated by the activity of Aurora A, comprising administering to the patient a therapeutically effective amount of the polymorph form of the claim 36; wherein, the condition mediated by the activity of Aurora A is cancer.

59. The method of claim 58, wherein the condition mediated by the activity of Aurora A is small cell lung cancer, colorectal cancer, gastric cancer, prostate cancer, breast cancer, triple-negative breast cancer, cervical cancer, head and neck cancer, esophageal cancer, ovarian cancer, thyroid cancer, non-small cell lung cancer, neuroblastoma, non-Hodgkin lymphoma, or any of combination thereof.

60. The method of claim 58, wherein the condition mediated by the activity of Aurora A is selected from small cell lung cancer, breast cancer, triple-negative breast cancer, cervical cancer, neuroblastoma or head and neck cancer.
